(19) United States
(12) Patent Application Publication
(10) Pub. No.: US 2009/0326333 A1
(43) Pub. Date: Dec. 31, 2009

(54) ASSESSING OUTCOMES FOR BREAST CANCER PATIENTS

(76) Inventors: Matthew P. Goetz, Rochester, MN (US); Fergus J. Couch, Rochester, MN (US); James N. Ingle, Rochester, MN (US); Vera J. Suman, St. Charles, MN (US)

Correspondence Address: FISH & RICHARDSON P.C., PO BOX 1022, MINNEAPOLIS, MN, 55440-1022, US

(21) Appl. No.: 12/096,713

(22) PCT Filed: Dec. 8, 2006
(86) PCT No.: PCT/US06/47070

§ 371 Date: Oct. 14, 2008

Related U.S. Application Data
(60) Provisional application No. 60/748,994, filed on Dec. 9, 2005.

(51) Int. Cl. A61B 5/00

(52) U.S. Cl. 600/300

(57) ABSTRACT

This document provides methods and materials related to assessing the likely outcome for mammals (e.g., humans) with cancer (e.g., breast cancer). For example, methods and materials for using the ratio of HOXB13 polypeptide expression to IL-17BR polypeptide expression to determine the likelihood of a breast cancer patient to experience breast cancer relapse or death are provided.

### BACKGROUND

1. Technical Field

This document relates to methods and materials involved in assessing the outcome of cancer patients.

2. Background Information

In the adjuvant treatment of estrogen receptor (ER) positive breast cancer, hormonal therapy reduces the risk of breast cancer recurrence and decreases mortality. Tamoxifen, one of the most commonly used medications in the adjuvant treatment of ER positive breast cancer, is a selective ER modulator that competes with estrogen for binding to the ER. When administered to women with surgically treated ER positive breast cancer, tamoxifen reduces the risk of recurrence and death when taken for five years.

The ER and progesterone receptor are the most important tumor markers that predict response to tamoxifen (Bardou et al., J. Clin. Oncol., 21(10):1973-9 (2003)). However, because a significant proportion of ER positive breast cancers fail to respond or eventually develop resistance to tamoxifen, additional prognostic markers including tumor size, tumor grade, and nodal status are commonly used by physicians to make treatment decisions. Clinical studies have demonstrated, however, that even in “good prognosis” tumors (e.g., estrogen positive, lymph node negative), up to 20% of women will experience recurrence despite 5 years of adjuvant tamoxifen therapy (Fisher et al., J. Natl. Cancer Inst., 89(22):1673-82 (1997) and Fisher et al., Lancet, 364(9437):858-68 (2004)). These findings indicate the need for additional markers that will identify women at high risk for recurrence.

### SUMMARY

This document provides methods and materials related to assessing the likely outcome for mammals (e.g., humans) with cancer (e.g., breast cancer). For example, this document provides methods and materials that involve using the ratio of HOXB13 polypeptide expression to IL-17BR polypeptide expression to determine the likelihood of a breast cancer patient to experience breast cancer relapse or death. As described herein, a high HOXB13 to IL-17BR expression ratio is associated with increased relapse and death in patients with resected node negative, ER positive breast cancer treated with tamoxifen and may identify patients in whom alternative therapies should be studied.

In general, one aspect of this document features a method for assessing the likelihood of cancer relapse. The method comprises determining whether or not a node-negative, ER-positive breast cancer patient contains cancer tissue with a HOXB13:IL17BR expression ratio indicative of an increased likelihood of experiencing cancer relapse, wherein the presence of said HOXB13:IL17BR expression ratio indicates that said patient is likely to experience cancer relapse, and wherein the absence of said HOXB13:IL17BR expression ratio indicates that said patient is likely to experience a relapse-free survival or a disease-free survival. The patient can contain cancer tissue with said HOXB13:IL17BR expression ratio, and the method can comprise classifying the patient as being likely to experience cancer relapse. The patient can not contain cancer tissue with the HOXB13:IL17BR expression ratio, and the method can comprise classifying the patient as being likely to experience a relapse-free survival or a disease-free survival.

In another embodiment, this document features a method for assessing the likelihood of cancer relapse. The method comprises, or consists essentially of, determining whether or not a node-negative breast cancer patient contains cancer tissue with a HOXB13:IL17BR expression ratio greater than −1.339, wherein the presence of the HOXB13:IL17BR expression ratio greater than −1.339 indicates that the patient is likely to experience cancer relapse, and wherein the absence of the HOXB13:IL17BR expression ratio greater than −1.339 indicates that the patient is likely to experience a relapse-free survival or a disease-free survival.

In another embodiment, this document features a method for assessing the likelihood of cancer survival. The method comprises, or consists essentially of, determining whether or not a node-negative breast cancer patient contains cancer tissue with a HOXB13:IL17BR expression ratio less than −1.339, wherein the presence of the HOXB13:IL17BR expression ratio less than −1.339 indicates that the patient is likely to experience longer survival than a comparable node-negative breast cancer patient who contains cancer tissue with a HOXB13:IL17BR expression ratio greater than −1.339.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting Kaplan-Meier Estimates of Relapse-Free Survival (RFS) in the entire cohort by HOXB13:IL-17BR (> vs. <−1.849).

FIG. 2 is a graph plotting Kaplan-Meier Estimates of Disease-Free Survival (DFS) in the entire cohort by HOXB13:IL-17BR (> vs. <−1.849).

FIG. 3 is a graph plotting Kaplan-Meier Estimates of Overall Survival (OS) in the entire cohort by HOXB13:IL-17BR (> vs. <−1.849).

FIG. 4 provides Hazard Ratios for Relapse-Free Survival (RFS), Disease-Free Survival (DFS), and Overall Survival (OS) by univariate Cox proportional hazard modeling, univariate Faraggi-Simon cross validation, multivariate Cox proportional hazards modeling, and Faraggi-Simon multivariate cross validation in all patients using HOXB13:IL-17BR (> vs. <−1.849).

FIG. 5 is a graph plotting Kaplan-Meier Estimates of Relapse-Free Survival (RFS) in the node negative cohort by HOXB13:IL-17BR (> vs. <−1.339).

FIG. 6 is a graph plotting Kaplan-Meier Estimates of Disease-Free Survival (DFS) in the node negative cohort by HOXB13:IL-17BR (> vs. <−1.339).

FIG. 7 is a graph plotting Kaplan-Meier Estimates of Overall Survival (OS) in the node negative cohort by HOXB13:IL-17BR (> vs. <−1.339).

FIG. 8 provides Hazard Ratios for Relapse-Free Survival (RFS), Disease-Free Survival (DFS), and Overall Survival (OS) by univariate Cox proportional hazard modeling, univariate Faraggi-Simon cross validation, multivariate Cox proportional hazards modeling, and Faraggi-Simon multivariate cross validation in node negative patients using HOXB13:IL-17BR HOXB13:IL-17BR (> vs. <−1.339).

### DETAILED DESCRIPTION

This document provides methods and materials related to assessing the likely outcome for mammals (e.g., humans) with cancer (e.g., breast cancer). Measuring the levels of HOXB13 and IL-17BR expression in a cancer sample, and using those levels to calculate an HOXB13:IL-17BR ratio, can help clinicians assess the likely outcome for the mammal (e.g., human) with cancer (e.g., breast cancer).

The ratio of HOXB13 expression to IL-17BR expression can be used to assess the likelihood of cancer survival and to assess the likelihood of cancer relapse. An HOXB13:IL-17BR ratio can be calculated from polypeptide or mRNA levels of HOXB13 and IL-17BR measured in a cancer sample or in other biological samples from a cancer patient. For example, an HOXB13 mRNA level and an IL-17BR mRNA level can be used to calculate an HOXB13:IL-17BR ratio. If an HOXB13:IL-17BR ratio is higher than a corresponding control HOXB13:IL-17BR ratio or cut-off ratio, then the patient can be classified as being likely to experience cancer relapse. If an HOXB13:IL-17BR ratio is lower than a corresponding control HOXB13:IL-17BR ratio or a cut-off ratio, then the patient can be classified as being likely to experience longer survival than a comparable cancer patient (e.g., a comparable node-negative breast cancer patient) who contains cancer tissue with a HOXB13:IL17BR expression ratio greater than the corresponding control HOXB13:IL-17BR ratio or cut-off ratio. In some cases, a cut-off ratio can be −1.339. A corresponding control HOXB13:IL-17BR ratio can be determined as described herein.

Any sample can be used when measuring the levels of HOXB13 and IL-17BR mRNA or polypeptides. Such samples include, without limitation, tissue biopsies (e.g., breast tissue biopsy), surgical waste, and isolated cells. When measuring HOXB13 and IL-17BR expression levels, suitable samples can include breast cancer samples. A sample can be manipulated prior to measuring HOXB13 and IL-17BR expression levels. For example, a breast tissue biopsy sample can be treated such that total mRNA is obtained. In another example, a breast cancer biopsy can be frozen, embedded, sectioned, and stained to identify cancerous regions.

Any method can be used to measure HOXB13 and IL-17BR expression levels in a sample. For example, methods for measuring HOXB13 and IL-17BR mRNA levels include, without limitation, PCR-based methods and in situ hybridization. In some cases, RT-PCR can be used with oligonucleotide primers designed to amplify HOXB13 and IL-17BR mRNA from a breast cancer sample. Once amplified, the products corresponding to HOXB13 and IL-17BR mRNA can be separated by gel electrophoresis, and the levels of HOXB13 and IL-17BR product determined by densitometry. The levels determined by densitometry can be used to calculate an HOXB13:IL-17BR ratio. The level of HOXB13 and IL-17BR mRNA also can be measured in a sample by in situ hybridization. For example, breast tissue samples can be collected and frozen. Adjacent sections prepared from the frozen breast tissue samples can be hybridized with biotinylated oligonucleotide HOXB13 probes to localize HOXB13 mRNA and biotinylated oligonucleotide IL-17BR probes to localize IL-17BR mRNA. Other sections can be hybridized with a control probe. Hybridizations to localize HOXB13 and IL-17BR expression can be performed under moderate to high stringency conditions. Moderate and high stringency conditions are well known in the art. For example, moderate stringency conditions can include hybridizing at about 37° C. in a hybridization solution containing 4×SSC, 50% deionized formamide, 10% dextran sulphate, 1×Denhardt's solution, 0.025% yeast tRNA, 0.05% denatured salmon testis DNA, 1 mM ribonucleoside vanadyl complexes, 3 ng/mL oligodeoxythymidine-12 mers, and 10 to 15 ng/μL of biotinylated probes, while the washes are performed at about 37° C. with a wash solution containing 4×SSC and 50% formamide solution. High stringency conditions can include hybridizing at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate. Hybridization conditions can be adjusted to account for unique features of the nucleic acid molecule, including length and sequence composition. Probes can be labeled (e.g., fluorescently) to facilitate detection. The distribution of HOXB13 and IL-17BR probes can be quantitated using an image analysis system. Breast tissue sections treated with RNAse before hybridization or incubated with a control probe can be used as controls to confirm that localization of HOXB13 and IL-17BR probes is specific. The levels of HOXB13 and IL-17BR mRNA quantitated by image analysis can be used to an HOXB13:IL-17BR ratio.

Methods for measuring polypeptide levels include, without limitation, ELISA-, immunohistochemistry-, and immunofluorescence-based techniques. HOXB13 and IL-17BR polypeptide levels in a breast tissue sample can, for example, be measured using a quantitative sandwich ELISA technique. Breast tissue samples can be homogenized and extracted, and aliquots of the extracts added to separate wells of a microtiter plate pre-coated with antibodies specific for HOXB13 or IL-17BR. After protein binding and subsequent washing, enzyme-linked antibodies (e.g., mouse monoclonal antibodies) specific for HOXB13 or IL-17BR polypeptides can be added to the wells. After antibody binding and subsequent washing, a substrate solution containing a label-conjugated anti-mouse IgG can be added to the wells (e.g., horseradish peroxidase (HRP)-conjugated anti-mouse IgG). The label then can be quantitated by spectrophotometry, and the quantitated levels used to determine an HOXB13:IL-17BR ratio.

In some cases, starting material containing both cancerous and normal tissues can be enriched for the cancerous portions. For example, cancerous breast cells within a breast tissue sample section can be individually collected using laser capture microdissection. The collected cancerous cells then can be homogenized, extracted, and processed by quantitative sandwich ELISA.

Polypeptide levels also can be measured by immunohistochemistry. For example, a breast tissue sample section can be treated with anti-HOXB13 primary antibodies, while an adjacent section can be treated with anti-IL-17BR primary antibodies. Negative control sections can be incubated with pre-immune rabbit or mouse serum in lieu of the primary antibodies. After antibody binding and subsequent washing, the primary antibodies can be detected with appropriate label-conjugated secondary antibodies (e.g., gold-conjugated or enzyme-conjugated antibodies). The label is then developed and quantitated using an image analysis system. The resulting quantitated polypeptide levels can be used to calculate an HOXB13:IL-17BR ratio.

Immunofluorescence techniques represent another approach to measuring the level of a polypeptide. For example, HOXB13 and IL-17BR polypeptides can be localized in the same breast tissue sample section using polyclonal or monoclonal antibodies against HOXB13 and IL-17BR. The bound antibodies can be detected using different fluorescently-conjugated antibodies. The levels of HOXB13 and IL-17BR fluorescence can be quantitated using an image analysis system, and the quantitated levels can be used to calculate an HOXB13:IL-17BR ratio.

Suitable antibodies for ELISA-, immunohistochemistry- and immunofluorescence-based methods can be obtained using standard techniques. In addition, commercially available antibodies to HOXB13 and IL-17BR can be used.

This document also provides kits that can be used to determine the levels of HOXB13 and IL-17BR mRNA or polypeptides in a sample. A kit provided herein can contain oligonucleotide primers or probes for detecting HOXB13 or IL-17BR nucleic acids, or antibodies for detecting HOXB13 or IL-17BR polypeptides. HOXB13 and IL-17BR antibodies that are components of the kits provided herein typically have specific binding affinity for either HOXB13 or IL-17BR. “Specific binding affinity” as it relates to an antibody describes an antibody's ability to interact specifically with a particular polypeptide without significantly cross-reacting with other different polypeptides in the same environment. An antibody having specific binding affinity for HOXB13 can interact with HOXB13 polypeptides specifically in the presence of multiple different polypeptides. The kits provided herein also can contain a reference chart that indicates a reference level for HOXB13 and IL-17BR polypeptides or mRNAs. Kits can be configured in any type of design (e.g., microtiter plate design) and can be made of any type of material (e.g., plastic).

The invention will be further described in the following examples, which do not limit the scope of the invention described in the claims.

### EXAMPLES

### Example 1

### Assessing Breast Cancer Outcomes

### Materials and Methods

Patients. The North Central Cancer Treatment Group conducted a randomized phase III clinical trial in postmenopausal women with resected ER positive breast cancer to assess the value of adding one year of fluoxymesterone to 5 years of tamoxifen adjuvant therapy (NCCTG 89-30-52; Ingle et al., Proc. Am. Soc. Clin. Oncol., 19:86a (2000)). Postmenopausal women with node-negative disease were required to have a stage T₁c or T₂N₀M₀ and could be any age, whereas women with node-positive disease were required to be at least 65 years of age with a tumor stage T₁-₂ N₁M₀. A woman was classified as postmenopausal if one of the following held: (1) her last menstrual cycle was >12 months prior to diagnosis, (2) her last menstrual cycle was 4-12 months prior to diagnosis and her follicle-stimulating hormone (FSH) level in the postmenopausal range, (3) she had a bilateral oophorectomy at least 2 months prior to diagnosis or (4) she had a hysterectomy without oophorectomy and was either >60 years old or her FSH level was in the postmenopausal range. Patients were surgically treated with either a modified radical mastectomy or breast conservative therapy including lumpectomy, axillary nodal dissection, and radiation therapy. The axillary dissection must have involved at least levels I and II and the examination of at least 6 axillary nodes. Patients who underwent lumpectomy must have had a primary tumor no larger than 5 cm, and the surgical margins must have been microscopically free of tumor. Post lumpectomy radiation therapy consisted of a total cumulative breast dose of 5040 cGy in 28 fractions, and those with axillary nodal involvement also received radiation to the axilla and supraclavicular regions. Patients were classified as ER positive if ≧10 fmol/mg cytosol protein or positive by an immunohistochemical assay. All patients were randomized within 6 weeks of definitive surgery.

A total of 541 patients were randomized to either oral tamoxifen, 20 mg daily for 5 years (256 eligible) or tamoxifen, 20 mg daily for 5 years plus oral fluoxymesterone, 10 mg twice daily for one year (258 eligible). Patients were stratified based on axillary lymph node status (0, 1-3, 4-9 vs. 10 or more), age (<65 years vs. >65), primary tumor size (<3 cm vs. >3 cm), ER status (10-49 fmol vs. 50 fmol or greater vs. positive by immunohistochemical assay), and extent of surgery (mastectomy vs. breast conservation therapy).

Clinical evaluations including history, physical examination, blood and chemistry groups, chest x-ray, and toxicity assessments were performed every 4 months for the first year, every 6 months years 2-5, and then yearly. Mammograms and pelvic examinations were performed annually. Toxicities were graded using the NCI Common Toxicity Criteria version 1.0 and the NCCTG supplement.

Within 30 days of registration, a paraffin embedded tumor block was submitted for fixture research purposes.

Tissue Preparation and RNA amplification. Utilizing the available 227 paraffin-embedded tissue blocks for the women enrolled in the tamoxifen only arm, one hematoxylin and eosin slide and two 7 mm sections were cut from each paraffin embedded block, and mounted on PEN membrane glass slides (Microdissect Gmbh, Germany), and stained using the Histogene™ Staining kit (Arcturus Bioscience, CA). Two breast cancer pathologists independently verified the presence of invasive breast cancer in 211 of the 227 paraffin blocks and provided Nottingham tumor grade for both invasive ductal and lobular carcinomas. In the case of assigning a score for tubule formation, the pathologist automatically assigned lobular carcinomas a score of 3 (<10% tubule formation). Tumor regions were isolated using laser-cutting and subsequently captured by using the Arcturus Veritas™ Laser Microdissection System (Arcturus Bioscience, CA) and immediately placed in proteinase K buffer (Paradise™ Reagent System, Arcturus Bioscience, CA). The proteinase K lysates were incubated for 16 hours at 50° C., and total RNA was purified and subjected to two rounds of linear amplification as described by manufacturer (Paradise™ Reagent System) to obtain amplified RNA (aRNA).

Real-time PCR. TaqMan® primers and probes were designed using Primer Express (Applied Biosystems, CA). The primer sequences for HOXB13 were 5′-GCCATGATCGTTAGCCTCATATT-3′ (forward primer; SEQ ID NO:1) and 5′-CAATTCATGAAAGCGGTTTCTAAAG-3′ (reverse primer; SEQ ID NO:2) with a minor groove binder (MGB) probe sequence VIC-TCTATCTAGAGCTCTGTAGAGC-MGB (SEQ ID NO:3); the primer sequences for IL-17BR were 5-GGCTTCCTA-TCCCACCAATT-3′ (SEQ ID NO:4) and 5′-AGGCTGTTTGTAGGCTGCA-3′ (SEQ ID NO:5) with an MGB probe sequence VIC-CAGGGAAAAAACGTGTGATG-MGB (SEQ ID NO:6).

An aliquot (200-500 ng) of the amplified RNA (aRNA) from each tumor sample was converted into cDNA via reverse transcription using the Paradise Reagent System. TaqMan® assays using 1/30th of the reverse transcribed material were performed in duplicate in 20 μL in a 384-well plate using the ABI 7900HT instrument (Applied Biosystems, CA); the samples were heated to 50° C. for 2 minutes, 95° C. for 10 minutes, followed by then 45 cycles of 95° C. 15 sec and 60° C. for 1 minute. For each gene, a standard curve with cDNA dilutions derived from amplified human universal total RNA (Stratagene) was constructed to obtain relative expression levels (i.e., quantities) of HOXB13 and IL-17BR. The HOXB13:IL-17BR ratio was obtained as the difference of log 2-transformed quantities of HOXB13 and IL-17BR. No control genes were measured as the direct ratio calculation does not require a normalization factor. To control for plate to plate variation of PCR reaction, standard curves were run on each plate.

Study Design and End Points. One objective of this study was to examine the relationship between the HOXB13:IL-17BR expression ratio and clinical outcomes of relapse-free survival, disease-free survival, and overall survival. Relapse-free survival (RFS) was defined as the time from randomization to documentation of the first of the following events: any recurrence (local, regional or distant) of breast cancer, a contralateral breast cancer or death. When estimating the distribution of RFS, patients who developed a non-breast second primary cancer (other than squamous or basal cell carcinoma of the skin, carcinoma in situ of the cervix, or lobular carcinoma in situ of the breast) prior to the diagnosis of a breast event were censored on the day their second primary was diagnosed. Patients who were alive without a breast recurrence, contralateral breast cancer or a second non-breast primary cancer were censored at the date of their last disease evaluation. Disease-free survival (DFS) was defined as the time from randomization to documentation of the first of the following events: any recurrence (local, regional or distant) of breast cancer, a contralateral breast cancer, a second primary cancer, or death due to any cause. Patients who were alive without any of these events were censored at the date of their last disease evaluation. Overall survival (OS) was estimated as the time from registration to death due to any cause.

To assess whether clinical outcome differed with respect to the HOXB13:IL-17BR expression ratio, a minimum p-value approach was used to identify a cut-point for the HOXB13:IL-17BR expression ratio that best discriminates between those patients with a poor clinical outcome and those patients with a better clinical outcome. For each clinical outcome, this ‘optimal’ cut-point was sought from among the observed values of the expression ratio above the 10^{th} percentile and below the 90^{th} percentile of the expression ratio distribution. To account for multiple testing a correction to the p-value associated with the ‘optimal’ cut-point was employed as proposed by Lausen and Schumacher (Lausen and Schumacher, Biometrics, 1992:73-85 (1992)) and modified by Altman et al. (J. Natl. Cancer Inst., 86(11):829-35 (1994)). The resulting p-value, denoted as p_{cor}, and uncorrected log-rank p-value are reported. Because inclusion of a biomarker dichotomized at its ‘optimal’ cut-point, in a Cox regression analysis may inflate the effect (Lausen and Schumacher, Biometrics, 1992:73-85 (1992); Altman et al., J. Natl. Cancer Inst., 86(11):829-35 (1994); Hilsenbeck et al., Breast Cancer Res. Treat., 22(3):197-206 (1992); Simon and Altman, Br. J. Cancer, 69(6):979-85 (1994); and Faraggi and Simon, Stat. Med., 15(20):2203-13 (1996)), the cross-validation approach of Faraggi and Simon was used to obtain a point and interval estimate of the hazard of each clinical outcome for those with a high HOXB13:IL-17BR expression ratio relative to those with a low HOXB13:IL-17BR expression ratio.

Log rank tests and univariate Cox proportional hazard models were used to assess whether the distributions of RFS, DFS, or OS differed with respect to any one of the following factors: age 65 years or greater (yes vs. no), extent of surgery (mastectomy vs. breast conserving), estrogen receptor status (10-49 fmols vs. ≧50 fmols vs. positive by immunohistochemistry), number of positive nodes (represented as three indicator variables for 1-3, 4-9, and ≧10 positive nodes), tumor size 3 cm or greater (yes vs. no), Nottingham grade (3 vs. 1 or 2), Her2 expression (3+ vs. 0, 1+, or 2+), and prior exposure to exogenous estrogens (yes vs. no). For each clinical outcome, multivariate Cox proportional hazard modeling was performed to obtain a subset of the potential prognostic factors which provided an adequate fit to the data. Residual plots were examined. The likelihood ratio test was then applied to assess whether HOXB13:IL-17BR expression ratio dichotomized at its ‘optimal’ cut-point made a significant contribution to the model. The cross-validation approach (Faraggi and Simon, Stat. Med., 15(20):2203-13 (1996)) was used to assess the impact of expression ratio on RFS, DFS, or OS after known prognostic factors have been accounted for. Finally, the prognostic value of the two-gene expression ratio was assessed in the node negative and node positive breast disease cohorts separately, using the same analysis approach.

### Results

Characteristics of the Patients. Of the 256 eligible women enrolled to the tamoxifen only arm, 211 paraffin-embedded tumor blocks were available for RNA extraction. The relative expression levels of HOXB13 and IL-17BR were obtained for 206 of these 211 patients. Table 1 presents the pre-registration characteristics for patients with and without gene expression data. The overall patient characteristics were similar, although a higher percentage of patients with HOXB13/IL-17BR expression ratio data had a tumor size greater than 3 cm (24%) compared with the group without HOXB13/IL-17BR expression ratio data (10%).

TABLE 1
Pre-registration characteristics of the patients randomized to the
tamoxifen arm that did and did not have expression ratio data.

| | Women with | Women without |
|---|---|---|
| | expression ratio data | expression ratio data |
| | (n = 206) | (n = 50) |
| Race | | |
| Caucasian | 92% | 91% |
| Age | | |
| Median (range) | 68 (42-84) | 68 (48-87) |
| Operative procedure | | |
| Mastectomy | 83% | 74% |
| Breast Conservation | 17% | 26% |
| Number of Positive Nodes | | |
| 0 | 63% | 62% |
| 1-3 | 26% | 15% |
| 4-9 | 7% | 15% |
| 10+ | 4% | 6% |
| Tumor Size | | |
| <3 cm | 76% | 90% |
| ≧3 cm | 24% | 10% |
| ER status | | |
| 10-49 fmols | 20% | 20% |
| ≧50 fmols | 69% | 56% |
| Positive | 11% | 24% |
| HER2 | | |
| 0 | 11% | Not determined |
| 1 | 36% | |
| 2 | 34% | |
| 3 | 18% | |
| unknown | <1% | |
| Histology | | |
| Ductal | 86% | Not determined |
| Lobular | 10% | |
| Other | 4% | |
| Nottingham Tumor Grade | | |
| Grade 1 | 26% | Not determined |
| Grade 2 | 55% | |
| Grade 3 | 18% | |
| Unknown | <1% | |

For the group of patients with gene expression data available, the first documented event was as follows: local, regional or distant breast recurrence (39 patients), contralateral breast cancer (12 patients), a second non-breast primary cancer (13 patients), both a breast recurrence and a second non-breast primary cancer (1 patient), and death without a breast recurrence or second primary cancer (37 patients). At last follow-up, 104 women are alive without evidence of a breast event or second primary, 25 are alive following a breast event or second primary cancer, 29 died with disease recurrence, 8 died having developed a second primary cancer, 29 died of other causes, and 8 died of unknown causes. The Kaplan-Meier estimates for the 5 year RFS, DFS and OS were as follows: 75.6% (95% CI: 69.1-80.9%), 74.3% (95% CI: 67.7-79.7%), and 78.2% (95% CI: 71.9-83.3%). The median length of follow-up among the 129 patients still alive is 11.0 years (range: 5.7-13.6 yrs).

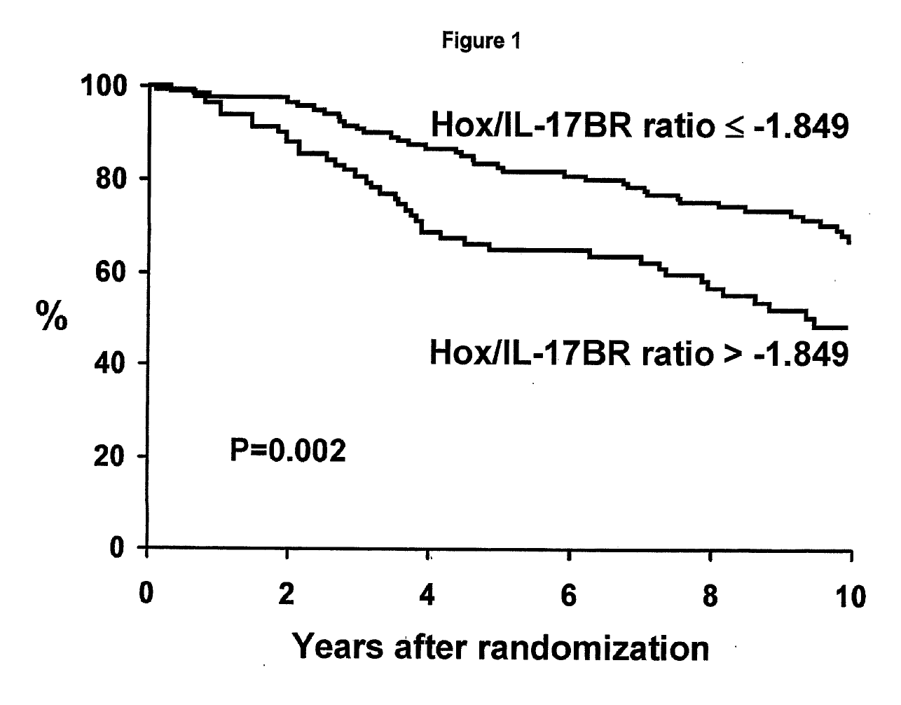

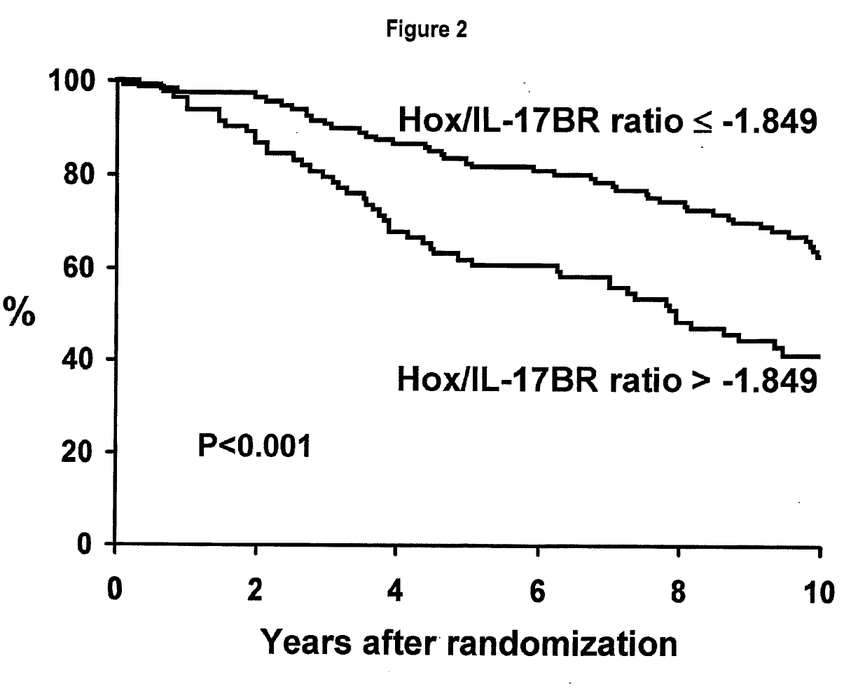

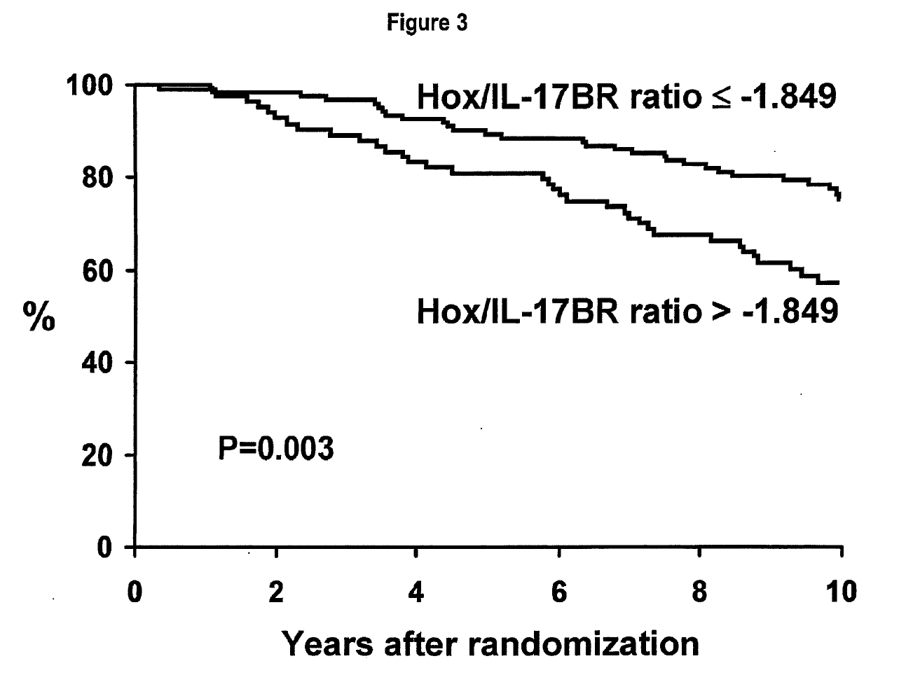

The HOXB13:IL-17BR expression ratio cut-point The cut-point for the log (HOXB13:IL-17BR expression ratio) that best discriminated clinical outcome (recurrence and survival) fell at the 58^{th} percentile of the observed HOXB13:IL-17BR expression ratio distribution (−1.849). This cut-off provided a classification which divided the women into two groups with significantly different DFS (p_{unc}<0.001), RFS (p_{unc}=0.002), and OS (p_{unc}=0.001). The Kaplan-Meier curves for RFS, DFS and OS using the cut point of −1.849 are shown in FIGS. 1-3. After applying the Altman method to correct for multiple testing, RFS (p_{cor}=0.044), DFS (p_{cor}<0.001), and OS (p_{cor}=0.025) still differed with respect to HOXB13:IL-17BR expression ratio cut point of −1.849. When using the Faraggi and Simon cross-validation method in the univariate Cox model, RFS (HR_{FS}=1.62, 95% CI: 1.06-2.48; p_{FS}=0.027), DFS (HR_{FS}=1.69, 95% CI: 1.14-2.51; p_{FS}=0.009), but not OS (HR_{FS}=1.55, 95% CI: 0.98-2.45; p_{FS}=0.060) differed with respect to HOXB13:IL-17BR expression ratio (< or >−1.849).

Assessing the added value of HOXB13:IL-17BR expression ratio. For each endpoint (RFS, DFS, and OS), Cox proportional hazard modeling was performed utilizing traditional patient and tumor prognostic factors. Nodal status (positive vs. negative), tumor size (≧3 cm vs. <3 cm), and Nottingham grade (3 vs. else) were significantly associated with each of these endpoints. When adjusting for these factors, women with a HOXB13:IL-17BR expression ratio >−1.849 disease had significantly worse RFS (HR 1.63, 95% CI: 1.05-2.53, p=0.030), DFS (HR 1.75, 95% CI: 1.16-2.63; p=0.008), and OS (HR 1.63, 95% CI: 1.63-2.60, p=0.041), independent of tumor size, nodal status and tumor grade, than women with a HOXB13:IL-17BR ratio <−1.849 (Table 2).

TABLE 2
Results of Cox modeling of RFS, DFS, and OS
in Entire Patient Cohort: Hazard Ratios (and
corresponding 95% confidence intervals).

| | Clinical Outcome | | |
|---|---|---|---|
| Factor | RFS | DFS | OS |
| Positive Nodes | 2.31 | 2.22 | 2.41 |
| | (1.50-3.54) | (1.49-3.31) | (1.54-3.79) |
| Tumor size | 1.93 | 1.98 | 2.01 |
| | (1.23-3.03) | (1.31-3.00) | (1.26-3.21) |
| Tumor Grade | 1.88 | 1.69 | 1.88 |
| | (1.13-3.14) | (1.04-2.75) | (1.11-3.18) |
| HOXB13:IL-17BR | 1.63 | 1.75 | 1.63 |
| expression ratio | (1.05-2.53) | (1.16-2.63) | (1.02-2.60) |

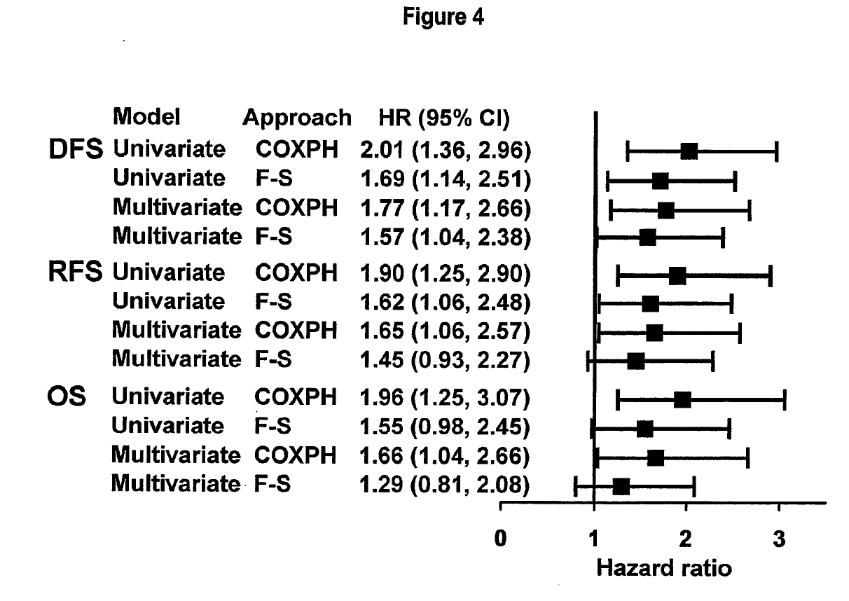

The Faraggi and Simon cross-validation method in the multivariate analysis was applied. It was found that although women with a HOXB13:IL-17BR expression ratio >−1.849, disease had significantly worse DFS (HR 1.57, 95% CI: 1.04-2.38; p=0.03) compared with a HOXB13:IL-17BR expression ratio <−1.849. There were no significant differences with respect to RFS (HR=1.45, 95% CI: 0.93-2.27; p=0.100) or survival (HR=1.29, 95% CI; 0.81-2.08; p=0.284) when tumor size, nodal status, and tumor grade were accounted for in this model. FIG. 4 shows a forest plot for each of the statistical analyses demonstrating the hazard ratio and corresponding 95% confidence intervals for RFS, DFS, and OS using the HOXB13:IL-17 cut point of −1.849.

The HOXB13:IL17BR expression ratio and nodal status. The distribution of the HOXB13:IL17BR ratio was assessed by nodal status. The median ratio was found to be similar when comparing node positive (median −3.81; range −10.15 to 9.37) with node negative patients (median −2.73; range −11.04 to 7.79), indicating that the assay performed well for both populations. Because the HOXB13 gene was previously determined to affect cell migration and invasion (Ma et al., Cancer Cell., 5(6):607-16 (2004)), the cut-point that best discriminated clinical outcome may differ as a function of nodal status. Therefore, the optimal cut points in the node negative (n=130) and node positive (n=96) cohorts were separately determined.

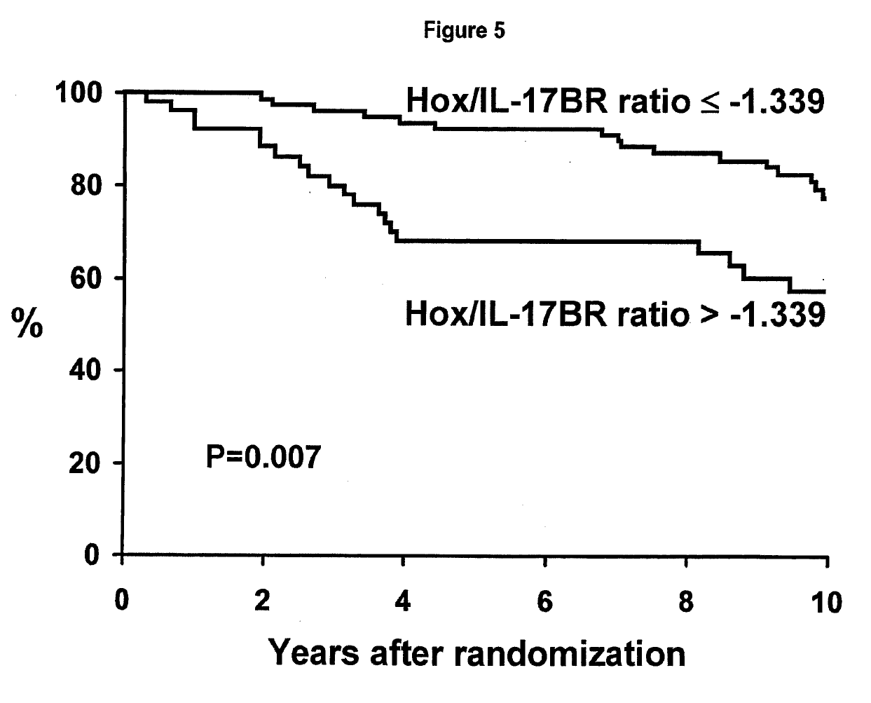

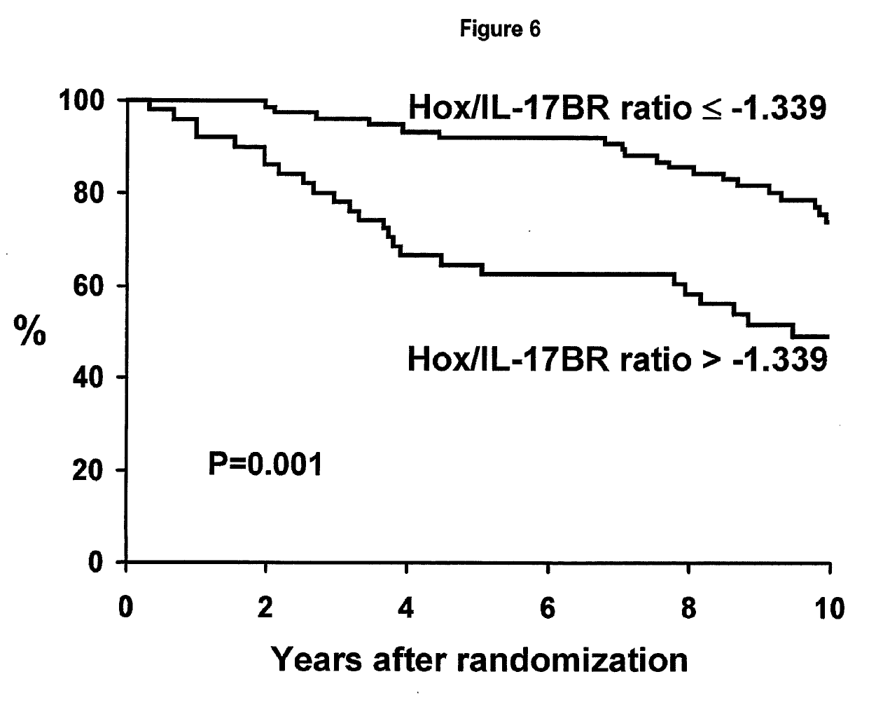

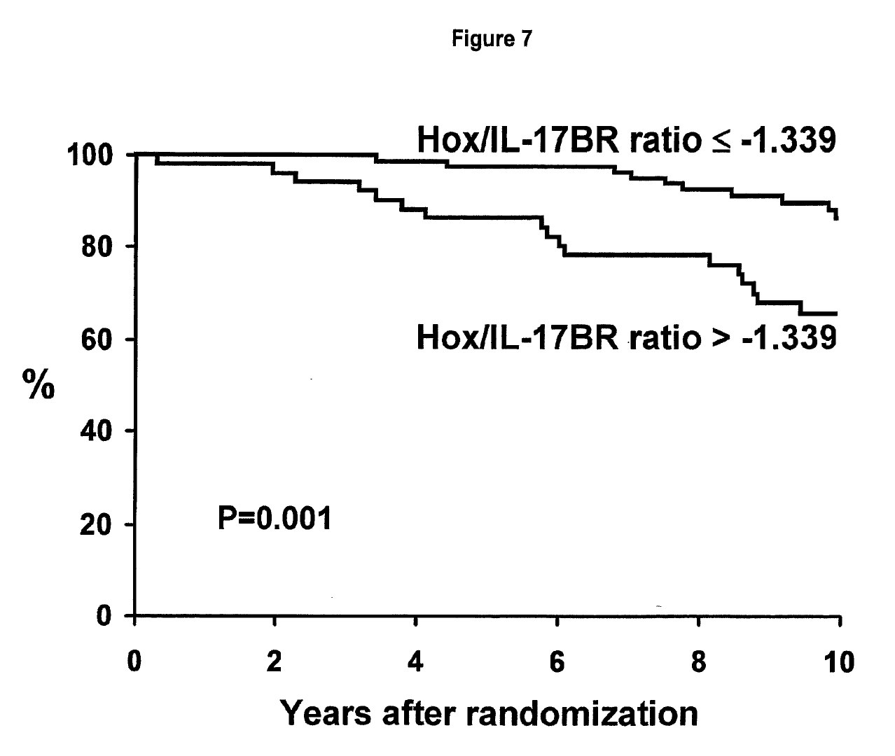

Node Negative Disease Among the 130 patients diagnosed with node negative disease, the ‘optimal’ cut-point fell at the 59^{th} percentile of the expression ratio distribution (−1.339). This cut-off provided a classification which divided the women into two groups with significantly different DFS (p_{unc}=0.001), RFS (p_{unc}=0.007), and OS (p_{unc}<0.001). The Kaplan-Meier curves are shown in FIGS. 5-7, and demonstrate that node negative patients with a HOXB13:IL17BR ratio of >−1.339 have significantly worse RFS, DFS, and OS compared with patients with a ratio of less than −1.339. After applying the Altman method to correct for multiple testing, DFS (p_{cor}=0.025), OS (p_{cor}=0.003), but not RFS (p=0.282), were found to differ with respect to HOXB13:IL17BR expression ratio cut point of −1.339. However, when the Faraggi and Simon cross-validation method was applied using a univariate Cox model, all three endpoints including RFS (HR_{FS}=1.99, 95% CI: 1.09-3.63; p_{FS}=0.025), DFS (HR_{FS}=2.12, 95% CI: 1.22-3.68; p_{FS}=0.008), and OS (HR_{FS}=2.35, 95% CI: 1.21-4.58; p_{FS}=0.012) differed with respect to HOXB13:IL17BR expression.

For each endpoint, Cox proportional hazard modeling was performed utilizing traditional patient and tumor prognostic factors in the node negative cohort. Only tumor size (≧3 cm vs. <3 cm) was significantly associated with each of these endpoints. The HOXB13:IL17BR expression ratio of −1.339 was used, and the likelihood ratio test was applied to assess whether the expression ratio was significantly associated with DFS, RFS, or OS. Patients with a HOXB13:IL17BR expression ratio >−1.339 had significantly worse RFS (HR 1.98, 95% CI: 1.07-3.68; p=0.031), DFS (HR 2.03, 95% CI: 1.15-3.59; p=0.015), and OS (HR 2.4, 95% CI: 1.19-4.84; p=0.014) independent of tumor size, compared with patients with a HOXB13:IL17BR expression ratio <−1.339 (Table 3).

TABLE 3
Results of Cox modeling of RFS, DFS, and OS in
Node-Negative Patient Cohort: Hazard Ratios (and
corresponding 95% confidence intervals).

| | Clinical Outcome | | |
|---|---|---|---|
| Factor | RFS | DFS | OS |
| Tumor size | 1.83 | 2.38 | 2.48 |
| | (0.92-3.65) | (1.30-4.36) | (1.22-5.06) |
| HOXB13:IL-17BR | 1.98 | 2.03 | 2.40 |
| expression ratio | (1.07-3.68) | (1.15-3.59) | (1.19-4.84) |

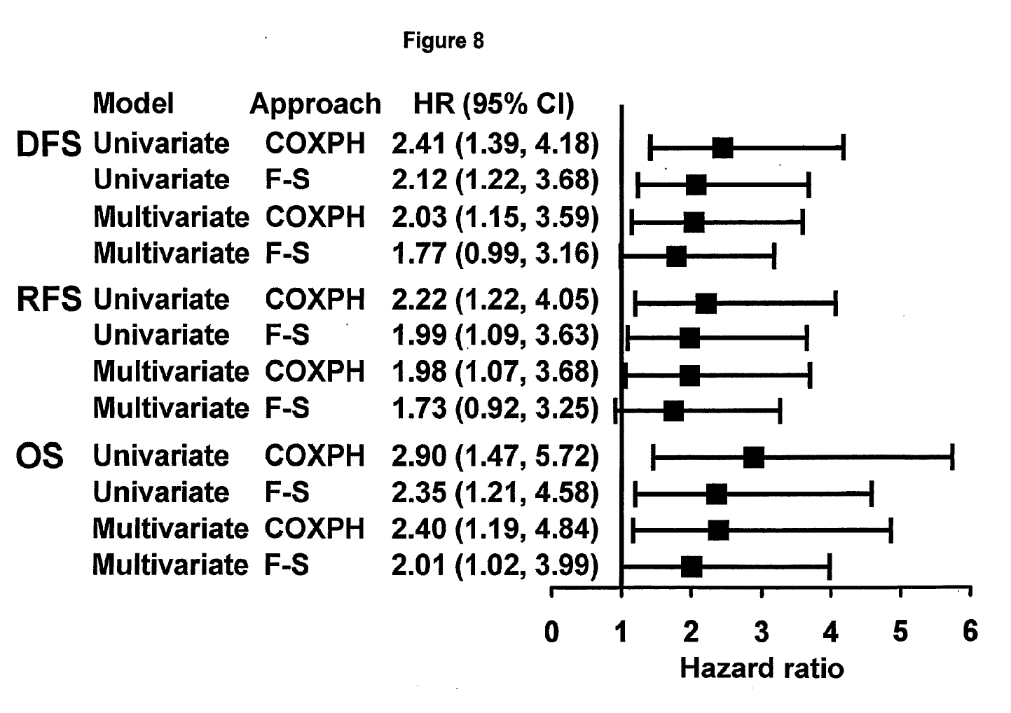

Finally, the Faraggi and Simon cross-validation method in the multivariate analysis was applied. Women with a HOXB13:IL17BR expression ratio >−1.339 tended to have worse RFS (HR=1.72, 95% CI: 0.92-3.25; p=0.088), DFS (HR=1.77, 95% CI: 0.99-3.16; p=0.054), and statistically significantly worse OS (HR=2.01, 95% CI 1.02-3.99; p=0.045), compared with patients with an expression ratio <−1.339. FIG. 8 shows a forest plot for each of the statistical analyses demonstrating the HR and corresponding 95% CI for RFS, DFS, and OS using the HOXB13:IL17BR cut point of −1.339.

Node Positive Disease. Among the 96 patients diagnosed with node positive disease, the ‘optimal’ cut-point was at the far right of the expression ratio distribution [namely, the 90^{th} percentile (4.4)]. Both the Altman approach and Faraggi and Simon procedure led to the conclusion that there was no evidence to suggest that RFS (p_{cor}=0.217, p_{FS}=−0.120), DFS (p_{cor}=0.148, p_{FS}=0.069), or OS (p_{cor}=0.148, p_{FS}=0.324) differs with respect to the HOXB13:IL17BR expression ratio.

The results provided herein by studying a cohort of postmenopausal women with tamoxifen treated breast cancer demonstrate that the HOXB13 to IL-17BR gene expression ratio is associated with breast cancer recurrence and survival, independent of standard clinical and pathological prognostic markers. Furthermore, these results demonstrate that this marker can be used in the node negative breast cancer patient population. Using statistical cross validation, it is demonstrated that only in the node negative cohort was a high HOXB13 to IL-17BR ratio associated with worse survival in the univariate (p<0.0001), the univariate Cox cross validation model (p=0.012), multivariate (HR 2.4, 95% CI 1.19-4.84, p=0.014), and multivariate cross validation analysis (HR 2.01; 95% CI 1.02-3.99; p=0.045).

The results that a high HOXB13 to IL-17BR expression ratio is associated with a greater risk of relapse and death in node negative ER positive breast cancer but not node positive suggest that this biomarker may be a marker of early invasion and metastatic potential. This notion is further supported by the fact that the recurrences seen in patients with a high HOXB13 to IL17-BR ratio occurred quickly, within the first four years of beginning tamoxifen, followed by a plateau until year 8, at which time further relapses were seen in both arms (FIG. 5). In women with negative lymph nodes and a HOXB13:IL-17BR ratio of less than −1.339, there were no events (recurrence or death) within the first 2 years after randomization. Therefore, the two gene expression ratio may identify the biologic underpinnings for the early peak in the hazard rate for relapse, typically seen 18-24 months following initiation of hormonal therapy (Baum et al., Proc. Am. Soc. Clin. Oncol., 23(16S):31S (2005)).

The demonstration that the two gene assay is associated with poorer outcomes in node negative ER positive breast cancer but not node positive breast cancer illustrates the complexity of performing biomarker studies in patients with breast cancer. Multiple gene expression profiling studies have been published which correlate a specific profile with breast cancer outcomes; however, some of these profiles were derived from patients treated with multiple different therapies (for example either chemotherapy alone or with/without hormonal therapy) for varying stages (I-III) of pre and postmenopausal estrogen positive and negative breast cancer. In contrast, the 2-gene profile was discovered and tested in patients with ER positive breast cancer treated with tamoxifen monotherapy. This point is important, because for ER positive breast cancers, it is less likely that clinicians will use gene expression profiling to exclude patients from hormonal therapy, given that hormonal therapy not only reduces the risk of distant recurrence, but also prevents the development of contralateral breast cancer (Howell et al., Lancet, 365(9453):60-2 (2005)). However, in the case of node negative ER positive breast cancer, the 2 gene biomarker may identify a high risk group of patients for which upfront aromatase inhibitors, and/or chemotherapy may prevent some of the immediate recurrences seen within the first 5 years with tamoxifen monotherapy. Although five years of tamoxifen remains the standard of care for the adjuvant treatment of pre-menopausal breast cancer, for postmenopausal women, the role of tamoxifen priming prior to the use of aromatase inhibitors is still being resolved.

The “cut point” generated and studied herein is different than that developed by Ma et al. (Cancer Cell., 5(6):607-16 (2004)) because of at least two reasons. First, new primers were designed for both HOXB13 and IL-17BR to improve PCR efficiency and precision using formalin fixed paraffin embedded samples that had been collected years earlier. Secondly, in contrast to the PCR values of HOXB13 and IL-17BR originally generated by Ma et al., which were z-transformed before taking the ratio and which required input normalization, the current data (derived from the NCCTG paraffin tumor blocks) were obtained without normalization genes and z-transform was not done. The resultant calculated ratio of HOXB13 over IL17BR is therefore on a different scale than the ratio obtained by Ma et al. Based on these differences in assay methodology and data analyses, a new cut-point needed to be established and “cross validated” using the statistical methods that were chosen.

In summary, the results provided herein demonstrate that the HOXB13 to IL-17BR gene expression ratio is associated with relapse and survival in node negative, but not node positive breast cancer. Studies using untreated breast cancer patients can confirm that the 2-gene expression ratio provided herein can be used as a prognostic marker. In addition, the 2-gene expression ratio provided herein can be used to determine whether alternative hormonal therapy (aromatase inhibitors) or chemotherapy will improve the outcomes of women identified to be at high risk by means of the HOXB13-IL-17BR ratio.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing the likelihood of cancer relapse, wherein said method comprises determining whether or not a node-negative, ER-positive breast cancer patient contains cancer tissue with a HOXB13:IL17BR expression ratio indicative of an increased likelihood of experiencing cancer relapse, wherein the presence of said HOXB13:IL17BR expression ratio indicates that said patient is likely to experience cancer relapse, and wherein the absence of said HOXB13:IL17BR expression ratio indicates that said patient is likely to experience a relapse-free survival or a disease-free survival.

2. The method of claim 1, wherein said patient contains cancer tissue with said HOXB13:IL17BR expression ratio, and said method comprises classifying said patient as being likely to experience cancer relapse.

3. The method of claim 1, wherein said patient does not contain cancer tissue with said HOXB13:IL17BR expression ratio, and said method comprises classifying said patient as being likely to experience a relapse-free survival or a disease-free survival.

4. A method for assessing the likelihood of cancer relapse, wherein said method comprises determining whether or not a node-negative breast cancer patient contains cancer tissue with a HOXB13:IL17BR expression ratio greater than −1.339, wherein the presence of said HOXB13:IL17BR expression ratio greater than −1.339 indicates that said patient is likely to experience cancer relapse, and wherein the absence of said HOXB13:IL17BR expression ratio greater than −1.339 indicates that said patient is likely to experience a relapse-free survival or a disease-free survival.

5. The method of claim 4, wherein said patient contains cancer tissue with a HOXB13:IL17BR expression ratio greater than −1.339, and said method comprises classifying said patient as being likely to experience cancer relapse.

6. The method of claim 4, wherein said patient does not contain cancer tissue with a HOXB13:IL17BR expression ratio greater than −1.339, and said method comprises classifying said patient as being likely to experience a relapse-free survival or a disease-free survival.

7. A method for assessing the likelihood of cancer survival, wherein said method comprises determining whether or not a node-negative breast cancer patient contains cancer tissue with a HOXB13:IL17BR expression ratio less than −1.339, wherein the presence of said HOXB13:IL17BR expression ratio less than −1.339 indicates that said patient is likely to experience longer survival than a comparable node-negative breast cancer patient who contains cancer tissue with a HOXB13:IL17BR expression ratio greater than −1.339.

8. The method of claim 7, wherein said patient contains cancer tissue with a HOXB13:IL17BR expression ratio less than −1.339, and said method comprises classifying said patient as being likely to experience longer survival than a comparable node-negative breast cancer patient who contains cancer tissue with a HOXB13:IL17BR expression ratio greater than −1.339.

9. The method of claim 7, wherein said patient does not contain cancer tissue with a HOXB13:IL17BR expression ratio less than −1.339, and said method comprises classifying said patient as being likely to experience shorter survival than a comparable node-negative breast cancer patient who contains cancer tissue with a HOXB13:IL17BR expression ratio less than −1.339.


SEQUENCE LISTING (6 sequences)

SEQ ID NO: 1 - DNA, 23 bp, Artificial Sequence
  Oligonucleotide
gccatgatcgttagcctcatatt

SEQ ID NO: 2 - DNA, 25 bp, Artificial Sequence
  Oligonucleotide
caattcatgaaagcggtttctaaag

SEQ ID NO: 3 - DNA, 22 bp, Artificial Sequence
  Oligonucleotide
tctatctagagctctgtagagc

SEQ ID NO: 4 - DNA, 20 bp, Artificial Sequence
  Oligonucleotide
ggcttcctatcccaccaatt

SEQ ID NO: 5 - DNA, 19 bp, Artificial Sequence
  Oligonucleotide
aggctgtttgtaggctgca

SEQ ID NO: 6 - DNA, 20 bp, Artificial Sequence
  Oligonucleotide
cagggaaaaaacgtgtgatg